(19) United States
(12) Patent Application Publication
(10) Pub. No.: US 2021/0298651 A1
(43) Pub. Date: Sep. 30, 2021

(54) System and Method for Analyte Monitoring and Predictive Modeling

(71) Applicant: Salvus, LLC, Valdosta, GA (US)

(72) Inventors: James LeFiles, Valdosta, GA (US); R. Clinton Beeland, Valdosta, GA (US); Ron Levin, Valdosta, GA (US)

(21) Appl. No.: 17/215,583

(22) Filed: Mar. 29, 2021

Related U.S. Application Data
(60) Provisional application No. 63/000,582, filed on Mar. 27, 2020.

(51) Int. Cl. A61B 5/145; A61B 5/00

(52) U.S. Cl. CPC A61B 5/14546; A61B 5/0004; A61B 2560/0431; A61B 5/6898; A61B 5/0022; A61B 5/7275

(57) ABSTRACT

A portable analyte detection, monitoring and modeling system and related methods are provided. The detection system includes a detector having one or more probes and associated detector circuitry that is in communication with a mobile device. The system is in communication with a server, where the detection system transmits analyte detection signals. The analyte detection signals are transmitted in real-time and the detection system receives analyte level information determined by the server. The server may process data from multiple probes to track multiple analytes or a single analyte based on the multiple different probe data from a single detector. Predictive modeling is implemented to signal remedial or preventative measures.

### CROSS REFERENCE TO RELATED APPLICATIONS

This application claims the benefit of U.S. Provisional Application No. 63/000,582, filed Mar. 27, 2020, the contents of which are hereby incorporated by reference.

### BACKGROUND

Analyte contamination is a problem for many industries, including the healthcare industry, and for the general public alike. Analytes such as pathogens may expand in a population rapidly. In some cases, pathogens may lay dormant or infect yet present in an asymptomatic carrier before an explosion of infections. There exists a need for a safe and effective system and method for analyte detection, monitoring and predictive modeling such that preventative measures may be implemented to slow or stop the spread of an analyte.

### SUMMARY

An analyte detection, monitoring and modeling system is provided. The system includes at least one portable detector having a probe and detector circuitry for detecting an analyte in a sample and producing analyte detection data; and at least one mobile device configured to wirelessly receive the analyte detection data from the portable detector and transmit the analyte detection data from the portable detector to a processor in real-time, wherein the processor is configured to: quantify a level of analyte; monitor the level of analyte; and display, in real-time, on the mobile device all data related to type and level of analyte present; and execute a predictive modeling system. According to one embodiment, the detector, mobile device and predictive modeling system are integrated into a single, mobile unit sized to be hand-held. According to one embodiment, the mobile device is a smartphone, tablet, or a portable computer. According to one embodiment, the at least one portable detector includes a plurality of probes, each of the probes configured to detect a different analyte. According to one embodiment, the at least one portable detector includes a collection apparatus configured to receive a target sample. According to one embodiment, the mobile device is configured to: wirelessly receive analyte detection data from the at least one portable detector for each of the plurality of probes and to transmit the analyte detection data from the at least one portable detector to a processor in real-time; and receive and display on the mobile device real-time analyte level data for each of a plurality of different analytes determined by the remote processor from the analyte detection data for the plurality of probes. According to one embodiment, the mobile device is configured to transmit a signal to initiate predictive modeling via the predictive modeling system. According to one embodiment, the predictive modeling system receives real-time analyte level data from the mobile device. According to one embodiment, the mobile device is configured to receive analyte detection data from two or more portable detectors. According to one embodiment, the predictive modeling system analyzes data for one or more variables such as personal health data, environmental data, weather data, analyte transmission rate, movement of vectors/carriers, building layout and air circulation.

A method of determining the level of analyte in a target sample and modeling future contamination is provided. The method includes the steps of:

introducing a probe of at least one portable detector system to a sample, wherein the probe is configured to detect at least one analyte in the sample;

wirelessly transmitting analyte detection signals from detection circuitry in communication with the probe to a mobile device of the at least one portable detector system;

transmitting, in real-time, the analyte detection signals from the mobile device to a processing system;

receiving, in response to the transmitted analyte detection signals, real-time analyte level data processed by the processing system from the analyte detection signals;

displaying the real-time analyte level data to a user on a display of the mobile device; and

predictively modeling future analyte spread, infection or contamination. According to one embodiment, the at least one portable detector system comprises a plurality of probes, each of the probes configured to detect a different analyte; and wherein the method further comprises concurrently transmitting analyte detection signals from detection circuitry in communication with each of the plurality of probes to the mobile device of the at least one portable detector system. According to one embodiment, the method includes the step of transmitting any modeling data to a third party capable of implementing remedial or preventative measures against the analyte contamination. According to one embodiment, the method further includes the step of producing a report including a prediction regarding future analyte spread, infection or contamination. According to one embodiment, the report includes one or more recommendations for preventing future analyte spread, infection or contamination. According to one embodiment, the method includes the step of denying or accepting the one or more recommendations by a user. According to one embodiment, the sample is taken from a surface, air, human or animal. According to one embodiment, the sample is taken from a public or private space selected from the group consisting of a food processing facility, healthcare facility, airport, train station, border crossing, and office space.

A kit is provided that includes an analyte detection, monitoring and modeling system as provided herein and at least one set of instructions.

### BRIEF DESCRIPTION OF THE DRAWINGS

FIG. 1 illustrates a system for real-time analyte detection, monitoring and predictive modeling system according to one embodiment.

FIG. 2 illustrates one embodiment of detector circuitry that may be implemented in the detector of FIG. 1.

FIG. 3 illustrates an embodiment of functional layers that may be implemented in the server of FIG. 1.

FIG. 4 is a diagram illustrating the types of data records that may be stored in the data storage layer of the server of FIG. 1.

FIG. 5 is a flow diagram of a method for detecting, monitoring and predictively modeling analyte levels in the system of FIG. 1.

FIG. 6 illustrates a mobile device of the system of FIG. 1 in one embodiment.

FIG. 7 illustrates a computer system which may be implemented in, or as, one or more parts of the system illustrated in FIG. 1.

### DETAILED DESCRIPTION

One or more aspects and embodiments may be incorporated in a different embodiment although not specifically described. That is, all aspects and embodiments can be combined in any way or combination. When referring to the systems and methods disclosed herein, the following terms have the following meanings unless indicated otherwise. The following definitions are meant to clarify, but not limit, the terms defined. If a particular term used herein is not specifically defined, such term should not be considered indefinite. Rather, terms are used within their accepted meanings.

As used herein, the term “analyte” refers to a substance that is detected, identified, measured or any combination thereof by the systems provided herein. The analyte includes any solid, liquid, or gas affecting (positively or negatively) a body of interest. The analyte includes, but is not limited to chemicals, microbes (beneficial or pathogenic), biomarkers, RNA, DNA, pathogen, antigen or portion thereof, antibody, virus (dead or alive), metabolite generated as a reaction to disease or infection, or viral protein. The virus proteins include the major structural proteins including spike, membrane, envelope and nucelocapsid which are commonly found on the surface of viruses. Particular examples of viruses include, but are not limited to, influenza virus (strain A or B), sever acute respiratory syndrome (SARS), SARS coronavirus (CoV) and SARS-CoV-2 (i.e., Covid-19).

As used herein, the term “pathogen,” “pathological contamination” and “pathological organism” refer to any bacterium, virus or other microorganism (fungi, protozoa, etc.) that can cause disease.

As used herein, the terms “target,” “sample” and “target sample” all refer to any matter (e.g., solid, liquid or gas) that may be subject to the methods and systems provided herein. Particularly, these terms refer to any matter (animate or inanimate) where analyte is capable of being detected and monitored. Suitable examples of targets include, but are not limited to, any animate or inanimate surface, soil, food, ambient air, laboratory, hospital, human (skin, hair or bodily fluid), animal (skin, hair or bodily fluid), an agricultural field, and any environmental location where analyte contamination is a concern.

As used herein, the term “modeling and “predictive modeling” refer to processes undertaken by the appropriate computer components (processors, servers, etc. of the predictive modeling system) to predict how an analyte may spread or infect a population or environment.

The factors leading to global epidemics and pandemics of analytes such as pathogens continue to increase. Such factors include population growth, global travel, and changes in age demographics. The disclosed systems and methods provide real-time and earlier detection of analytes in the environment and immediately dispenses test results to public health organizations or other authority to facilitate rapid modeling and effective response to minimize the impact. Technical solutions are provided herein that allow the determination of contamination in a short enough time, and allow informed decision making by people with no advanced pathological analytical knowledge.

Methods and systems are provided herein to address the need to monitor and perform tests as well as provide results in real-time. Additionally, methods and systems for using this real-time analyte detection to reliably track and verify a sample's exposure to analytes are disclosed. The methods and systems provided herein may also allow for the predictive modeling of how an analyte might spread and the impact of such spread in any population such as, for example, the plant and animal/human population. According to some embodiments, the systems provided herein may be deployed in public places such as transportation hubs (airports, train stations, bus stations, border crossings) in an effort to detect, monitor and predictively model a future analyte contamination. In some embodiments, if an unacceptable level of analyte is detected, one or more of the systems as provided herein may be mobilized to a particular location for more concentrated monitoring and predictive modeling.

According to one embodiment, the system provided herein is mobile or portable for ease of use on-site in various environments. The system may be hand-held. The system may include a variety of components as provided herein within a rugged, stable shell or case. The system may also be powered via alternating current or direct current. The direct current may be provided by a battery such as, for example, one or more lithium, alkaline, gel, or AGM batteries, including deep cycle batteries. The direct current may be provided by alternative sources such as wind or solar. The alternative sources may provide current directly or be stored in one or more appropriate batteries for later use.

The system may be equipped with one or more software packages loaded within. The software may be electronically connected to the various system components as provided herein. The software may also be electronically integrated with a display for viewing by a user. The display may be any variety of display types such as, for example, a LED-backlit LCD. The system may include a memory component such that operating instructions for the system may be stored and all data related to detected analyte levels may be stored or archived for later retrieval or downloading onto a workstation or smartphone.

According to one embodiment, the system may include a collection component. The collection component may include an inlet for sample collection (i.e., a solid, fluid, or air-based sample). The collection component may be a physical extension of sampling area with an electronic signal connection to a detector component as described herein. The collection component may include or be connectable to a probe designed to generate a signal when exposed to a specific analyte.

According to one embodiment, wherein the wireless signal is processed with specialized algorithms based on chemistry, physics, and/or quantum mechanics by a server such as a remote server and the output data is nearly instantaneously wirelessly transmitted back to the mobile system from the server certifying an acceptable level of analyte when achieved. According to one embodiment, the sensing unit is mobile and sized to be hand-held. According to one embodiment, current versions of the algorithms appropriate to the analytes being tested are loaded on the sensing unit to allow it to operate independently of wireless communications. The mentioned algorithm may include the ability to combine inputs from sensors based on differing technologies to identify substances that individual sensing technologies would typically not be able to distinguish.

According to one aspect, a method of determining and monitoring the level of analyte in a sample is provided. The method includes the steps of collecting a sample and detecting any analyte in the sample. According to one embodiment, the method further includes the step of transmitting a signal regarding the level of analyte in the sample to a device at a remote destination. The remote destination device may be a locally operated mobile or portable device, such as a smart phone, tablet device, pad, or laptop computer. In other embodiments, the remote destination may be a stand-alone or networked computer, cloud device, or server accessible via a local portable device. According to one embodiment, when the signal is transmitted wirelessly to a server (such as a remote server), a return signal is transmitted to the system providing certification when an acceptable level of analyte is achieved.

According to one embodiment, the system as provided herein includes a detector. The detector may utilize gold catalyzed chemiluminescence immunoassay, immunoassay in microfluidics, electropathological immunoassay, or dip-stick immunoassay. According to one embodiment, the detector may utilize an interferometric sensor based on a planar optical waveguide. According to one embodiment, the detector may utilize immunoassays on top of the waveguide for detection of one or more analytes. According to one embodiment, the detector may include one or more polymer(s). According to one embodiment, the detector may include, or function based on, an enzyme-linked immunosorbent assay. According to one embodiment, the detector may utilize or more polypeptides, nucleic acids, antibodies, carbohydrates, lipids, receptors, aptamer or ligands of receptors, aptamers, fragments thereof, and combinations thereof such as that set forth in U.S. Patent Pub. No. 20080138797, the entirety of which is hereby incorporated by reference herein.

According to one embodiment, the detector may provide a visible color change to identify a particular analyte. According to on embodiment, the detector may include a reference component that provides secondary confirmation that the system is working properly. Such secondary confirmation may include a visual confirmation or analyte reference that is detected and measured by the detector.

According to one embodiment, the detector includes at least one filter. The filter may be located between the collection and component and the detector. According to one embodiment, the at least one filter includes activated charcoal. According to one embodiment, the at least one filter includes at least one resin such as anion exchange resin, cation exchange resin, softener resin, or a combination thereof.

According to one embodiment, the detector analyzes a sample that may include one or more analytes that require detection and certification of a certain level. According to one embodiment, the detector is calibrated to detect certain levels of at least one analyte such as a pathogen. The detector may be sensitive down to a parts per million level. According to one, the detector may also be sensitive down to a parts per billion level. According to another embodiment, the detector may also be sensitive down to a parts per trillion level. The detector may be sensitive to analyte that is present in a sample at the decigram level or decigram per milliliter level. According to one, the detector may also be sensitive to analyte present in a sample at the centigram level or centigram per milliliter level. According to one, the detector may also be sensitive to analyte present in a sample at the milligram level or milligram per milliliter level. According to one, the detector may also be sensitive to analyte present in a sample at the microgram level or microgram per milliliter level. According to one, the detector may also be sensitive to analyte present in a sample at the nanogram level or nanogram per milliliter level. According to one, the detector may also be sensitive to analyte present in a sample at the picogram level or pictogram per milliliter level.

According to one embodiment, the detector is calibrated to detect certain levels of at least one analyte down the levels provided herein. By gathering and transmitting real-time sensor data from more than one type of probe, a computation layer of a server, such as in a remote server, in the disclosed system may use an algorithm to interpret the signals in direct real-time comparison for immediately identifying and quantifying the concentration of different analytes. In alternative embodiments, the system may make the comparisons, analysis, and calculations itself with or without the use of the processing power of the remote server. The remote server may or may not utilize relevant data and calculation techniques that are not available to the mobile device or detector system.

The sample introduced to the system described herein may be obtained from various sources. The source includes air and any surface that may have been in contact with a analyte. The system as provided herein may be placed in fluid communication with a sample so as to detect and certify acceptable analyte levels in real time. Fluid communication may be established via a tube or other conduit that allows any fluid containing at least one analyte to come in contact with, or flow through, the system as provided herein.

According to a particular embodiment, the source may be air surrounding a particular area where human or animal analyte contamination is a concern. The air may be in a public or private space. The air may also be indoors or outdoors. Exemplary indoor spaces include transportations hubs (airports, train stations, border crossings, etc.), hospitals, parks, schools, office spaces, and healthcare facilities. According to the various embodiments described herein, the system and method may signal the need for remedial measures (i.e., decontamination) to minimize the risk of spreading the analyte. According to one embodiment, the detector may be optionally equipped to analyze additional environmental factors such as, for example, particulate matter (viable and otherwise), temperature, air speed, geolocation and humidity. According to the various embodiments described herein, the system and method may reduce the time typically required for decontamination, minimize the need to utilize (and store) large volumes of cleaners (e.g., harsh chemicals), reduce dependency of the operator to execute decontamination processes without benefit of knowledge of the point completion, and/or reduce legal risk to the operator by providing documentation of decontamination for a particular person, animal, area or surface.

The system as provided herein may also include a transmitting component. The transmitting component may be in electronic signal communication with the detector component. The transmitting component sends or transmits a signal regarding real-time analyte level data. Such data may provide evidence of analyte removal and/or inactivation. The transmission of such data may include real-time transmission via any of a number of known communication channels, including packet data networks and in any of a number of forms, including text messages, email, and so forth. Such real-time transmission may be sent to a remote destination via a wireless signal. The wireless signal may travel via access to the Internet via a surrounding Wi-Fi network. The wireless signal may also communicate with a remote destination via Bluetooth or other radio frequency transmission. The remote destination may be a smart phone, pad, computer, cloud device, or server. The server may store any data for further analysis and later retrieval. The server may analyze any incoming data using artificial intelligence learning algorithms or specialized pathological, physical, or quantum mechanical expertise programed into the server and transmit a signal back to the system confirming an acceptable of analyte is present. According to one embodiment, the system or server may be equipped with, or have access to, analyte level reference data such that certification may be received by the system alerting a user that an acceptable level of analyte is present. An acceptable level of analyte may be any predetermined level that is set by a rule-making authority such as, for example, the Environmental Protection Agency (EPA), the Food and Drug Administration (FDA), or Occupational Safety and Health Administration (OSHA).

According to one embodiment, the system includes a wireless data link to a phone line. Alternatively, a wireless data link to a building Local Area Network may be used. The system may also be linked to Telephone Base Unit (TBU) which is designed to physically connect to a phone jack and to provide 900 MHz wireless communications thereby allowing the system to communicate at any time the phone line is available.

A method of determining the level of analyte in a sample is also provided. The method includes the step of collecting a sample. The method further includes the step of detecting any analyte in the sample. The method utilizes at least one detector as described herein which is in electronic communication with the transmitting component. The method further includes the step of displaying the analyte levels to a user of the system. The step of displaying the analyte levels may be carried out via projecting any real time data on a screen as described herein.

The method may further include the step of transmitting a signal regarding the level of analyte in the sample to a destination. The step of transmitting may occur via a wireless signal, Bluetooth, radio frequency, local area network, or via a traditional phone line. The signal from the system includes data related to the level of analyte in the sample and diagnostic information about the sensor and the parameters around its use. The destination may be smart phone, pad, computer, cloud device, or server. The destination may, in turn, communicate or signal the system that an acceptable level of analyte is achieved or that the level is unacceptable. In the event the level of analyte is acceptable, the destination may communicate a certification of acceptable analyte level. The certification may be based on environment standards promulgated by an authority such as, for example, the EPA, FDA or OSHA. The certification may also be simultaneously submitted to a local or national authority such as, for example, the Center for Disease Control (CDC), EPA, FDA or OSHA. According to an alternative embodiment, the destination is a smart phone, pad, computer, cloud device, or server under the custody of a local or national authority such as, for example, the EPA, CDC, OSHA or FDA.

The method may further include the step of disposing of the sample per legal requirements. Such legal requirements assure that any sample still containing unacceptable levels of analyte are disposed of properly so as not to cause harm to a user or the environment.

A method can be integrated with a process of predictively modeling how the analyte contamination may progress within an environment or population. The modeling may optionally be performed by a processor at a remote facility or may be performed within a handheld or mobile device as provided herein. The modeling data may be presented in a graph, table, map or other acceptable visual depiction. Any modeling data produced may be transmitted to a third party such as a government authority or the Centers for Disease Control and Prevention (CDC). By alerting the appropriate authority, remedial or even preventative measures may be taken such as, for example, decontamination or cleaning operations or the shut-down of operations in the target sample area (e.g., shelter in place). A step of contact tracing may also be initiated upon notification of the appropriate authority.

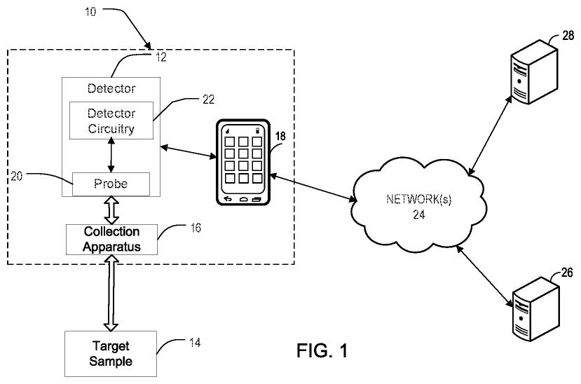

Referring to FIG. 1, an embodiment of a analyte detection system 10 is shown. The system 10, includes at least one detector unit 12, also referred to herein as a detector, configured to sample a test item 14 for a detection target, such as a analyte, via a collection apparatus 16. While not illustrated, more than one or a plurality of detector units 12 may be utilized together across a geographic region including across a field, city, county, state, country or around the globe. The collection apparatus 16 may be any of a number devices configured to route the analyte source or sample from the test item 14 into contact with the probe 20 of the detector 12. For example, the collection apparatus 16 may be a liquid conduit, or liquid conduit and pump arrangement when the test item is a liquid. Alternatively, the collection apparatus may be a gas conduit, or a fan and gas conduit if the test item is a gas or in the ambient air. The collection apparatus 16 may be integrated with the detector unit 12, or may be removable connectable to the probe 20 of the detector unit.

The at least one detector units may communicate the raw data or findings of the probe 20 in real-time with a mobile device 18. The mobile device 18 may receive the raw data or findings from one or more detector units 12 located in various geographic locations. The mobile device 18 may include logic stored in local memory on the mobile device to interpret the raw data and findings directly, or it may communicate over a network 24 with a remotely located server 26 to transfer the raw data or findings and request interpretation by logic located at the server 26. The mobile device 18 may be a handheld device, such as a smart phone, tablet, laptop computer that permits a user access to the real-time measurements of the probe and their real-time interpretation by a server 26 such as a remote server. As described in greater detail below, the real-time interpretation of analyte levels may be displayed to the user on the mobile device with an indication of whether the amount of analyte is in a desired range.

According to one embodiment, the analyte detection system 10 and all associated internal and display components are entirely handheld in a single unit. According to such an embodiment, the detector 12, detector circuitry 22, probe 20, collection apparatus, and mobile device 18 are contained within a single, mobile unit that can be held in one hand. According to such an embodiment, the mobile device 18 is a screen that may be operated via tactile buttons or via a touchscreen.

In some embodiments, the information received back from the server 26 may include notification that a modeling and potential remedial or preventative measure may be required. Additionally, the server, such as a remote server, may concurrently communicate results and modeling processes to a third party server 28 (such as the CDC or a governmental authority), insurer, or other interested party.

According to one embodiment, the detector 12 may be configured to look for a desired detection target and thus may be used to monitor or sample a desired substance for purity. The detector unit 12 may be configured to look for a particular analyte or contaminants.

The detector unit 12 may include a probe 20 in communication with detector circuitry 22. The probe 20 may be a single purpose probe 20 designed for detection of one type of analyte, may include a plurality of probes 20 each designed to detect a different respective analyte, or may include one or more probes 20 each designed for detection of more than one type of analyte. As will be evident in the examples provided below, the probe 20 may be placed in contact with, or proximity to, the target item being measured via the collection apparatus. The detector circuitry 22 may be configured to translate probe information into electrical signals or data in a predetermined format and to transmit the electrical signals or data over a wireless (e.g., Bluetooth) or wired connection to the mobile device. The detector circuitry may perform some or all of any data adjustment necessary for the sensed information from the probe 20, for example adjustments to the sensed information based on probe type or age, or may simply pass the data on for transmission to the mobile device 18.

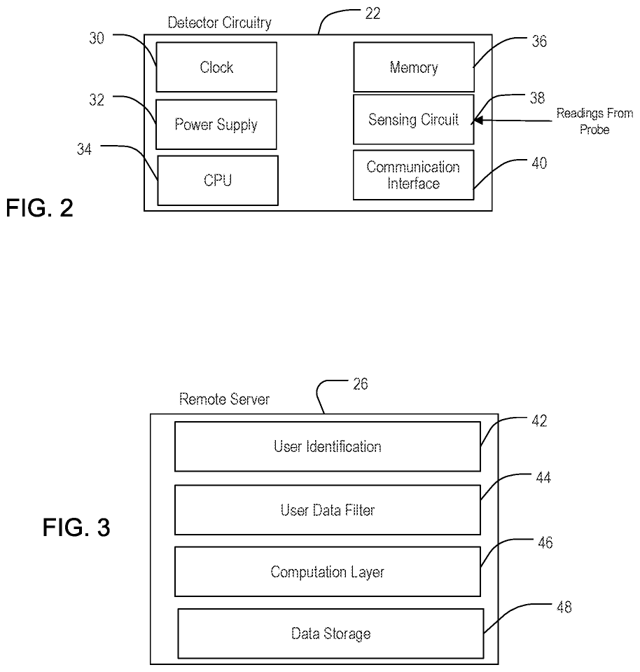

As illustrated in FIG. 2, an embodiment of the detector circuitry 22 is shown. The detector circuitry 22 included in the detector unit 12 may include a power supply circuit 32 (battery or AC), an internal clock 30 for tracking measurement times and dates for the associated probe 20, a sensing circuit 38 arranged to receive measurements or readings from the probe 20, and a communication interface 40 for communicating with the mobile device 18. The detector circuitry 22 may include a central processing unit (CPU) 34 or other controller, along with a memory 36 for storing executable instructions for operating the detector unit 12 and storing information sensed from the probe 20. The probe may include pathological, electrical, optical, and/or other sensitivity and is configured to translate the sensed information into electrical signals for the sensing circuit B5 to recognize. The CPU 34 may control the detector unit to transmit the data immediately from the sensing circuit 38 to the mobile device 18 via the communication hardware B6. Alternatively, the sensing circuit 38 may store the sensed information in the memory 36 and the CPU 34 may cause the sensed information to be transmitted at predefined intervals via the communication hardware 40. In yet other implementations, the CPU 34 may only direct the sensing circuit 38 to sample the probe 20 information at predetermined time intervals (e.g. a fixed number of milliseconds apart) and transmit the sensed information at the same, or a different, interval via the communication interface 40.

Referring to FIG. 3, the server 26 may be a computer configured as a web page host providing web-enabled services and including functional layers such as user identification management 42, a user data filter 44, a computation layer 46 and a data storage layer 48. The user identification management 42 may be a user authentication function to verify that authenticated users and mobile devices are properly screened and allowed access. The computation layer 46 may include functionality that receives raw or partially processed data from a detector 12 via a mobile device 18 and determines the type and level of analyte associated with the received data based on predetermined algorithms. Although the computation layer 46 functions of the server 26 may also, or alternatively, be stored in the mobile device 18. In certain embodiments, an advantage of real-time transmission of the detected data to the server 26 for processing is that greater processing power may be applied to more quickly translate the received data into analyte level determinations. The real-time transmission of data to the server 26 may also allow for modeling and comparison with other measurements of analyte at the same location, in the general area or around the globe. Also, the central location of the computation layer 46 in the remotely located server 26 provides a centralized location with which to update and control the techniques used to translate the data from the various detectors 12. In different implementations, the computation layer 46 may implement artificial intelligence learning algorithms or specialized pathological, physical, or quantum mechanical expertise programs to process the real-time data into analyte levels for immediate transmission from the server 26 to, and display on, the mobile device 18.

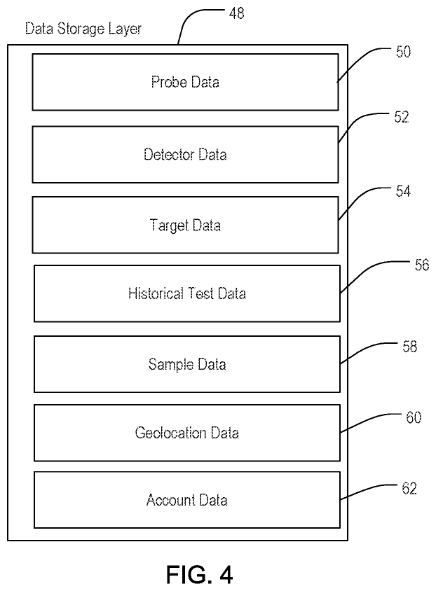

The data storage layer 48 may include data on users, devices, device types, and, as discussed in greater detail below, a history of analyte test results. Referring now to FIG. 4, an example of the data types stored in the data storage layer of the server, such as a remote server, is shown. The data storage layer may include probe data 50 for the various probes 20 that are associated with detectors 12 in the field and registered with the system. The probe data 50 may include information about each specific probe 20, such as the type and age of the probe (e.g. the number of tests run with the probe and the in service data of the probe). The probe data 50 may additionally include information on the probe's technology, including the substances testable by the probe alone or in combination with other probes, probe age calibration curves for use by the computation layer to adjust data received from the probe to account for potential effects of aging on the measurements, and probe technology interaction algorithms, for example this information may be an algorithm such as described herein to use multiple probe data received concurrently to differentiate for detection of a compound/analyte that may not be directly discernible by a single probe. Similarly, detector data on the detector 12 itself may be stored in the data storage layer 48 of the server 26. The detector data 52 may include serial number and MAC ID for the specific hardware, identification of authorized users, the location of the last use of the detector and the account ID associated with the detector 12. Data 54 that is descriptive of the target sample being tested and tracked may be included in the data storage layer 48 of the server 26. The data 54 may include the unique identifier of the target sample and the account identification (ID) of any account associated with the target sample or an account identification associated with the target sample itself. The data 54 may include geolocation data related to the location of the sample.

To provide improved tracking and certification of monitoring and the history of analytes, the data storage layer 48 also may include historical test data 56 received from different detectors 12 and associated with specific locations or test sample. The historical test data 56 may be stored at a remote location, directly on the mobile device, or both. The historical test data 56 may include data for each test run, such as: a record that probe compatibility was confirmed for each test, the time stamps and detector values received for the test, the age of probe corrections and probe interaction factors determined for the test, and the calculated values for the analyte. Additionally, historical test data 56 for each test run may include location and identification information, such as the geolocation of the detector 12 at time of test, the identifier information for the target sample, detector, user, and probe(s) 20 for that test run, and the account ID of the entity for whom the tests are being run and tracked. In order to link the individual tests to a sample, the historical test data 56 may also include data 58 for the particular sample tested, such as the time stamps of the test, the location, a bar code (or other unique identifier), and a test identifier number. When the testing is performed at a food processing plant, the server 26 may also include the lot number, food description and or food pack universal product code (UPC) or other identifier and link that to the history of testing of the food and analyte exposure of the food that went into that lot of processed food. Geolocation information 60 on the location at which testing has been or will be performed may also be stored in the data storage layer 48. The location information 60 may include geofencing coordinates, such as perimeter coordinates, along with a description of the area, location or environment. Account data 62 may be stored in the data storage layer as well, including user IDs and associated information associated with each account that utilizes the system.

Any of a number of probe types and technologies may be used in different embodiments. An example of a probe type that maybe used to differentiate between often difficult to differentiate analytes may include probes that are an interferometric biosensor type, such as an a molecularly imprinted polymer (MIP), antibody assay probes, aptamers, DNA, RNA or proteins. These probes may be part of a detection system 10 that produces real-time readings for which the rate of change of those readings output by the probes may be measured with the disclosed detection system 10. For example the probes may each generate a diffraction or interferometric pattern and the changes in that pattern are detected and analyzed by the computation layer or locally at the mobile device 18 of the detection system 10, and are translated into a analyte level, and not just a presence or absence of the analyte. In one implementation, the analyte level may be proportional to a rate of change of the diffraction pattern measured, such that an integration of the rate of change in the diffraction pattern may be used to determine concentration levels. This calculation may take place locally at the mobile device 18 or remotely at the server 26.

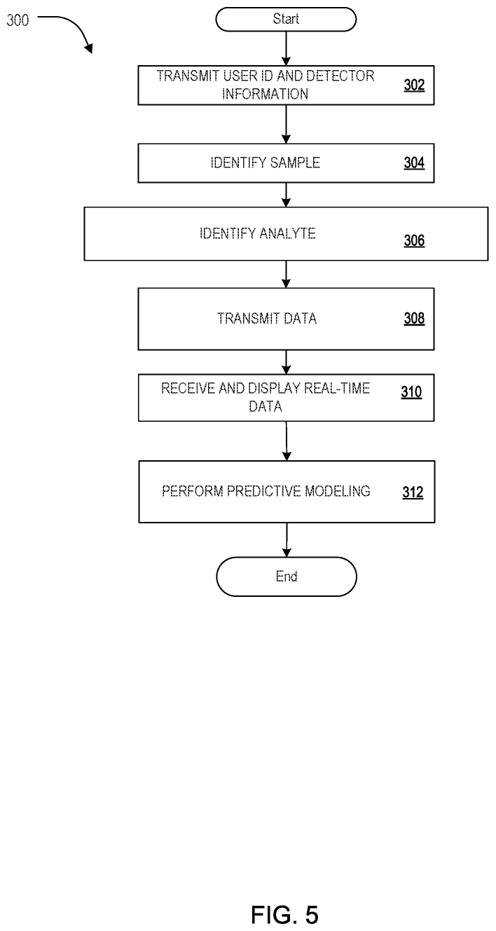

One embodiment of a method 300 using the systems described above is illustrated in FIG. 5. Using a handheld system such as illustrated in FIG. 1, the user may first enter a user identifier (ID) in the mobile device and the mobile device transmits that information to the server for authentication, along with automatically appending information on the detector 12, which may include probe and/or detection circuitry identifying information (at 302). The probe and/or detection circuitry identifying information may include serial number information for the probe 20 and detection circuitry 22, the Media Access Control (MAC) address for each and the Internet protocol (IP) network address. After receiving and transmitting data at the mobile device for authenticating the user, detector 12 and mobile device 18, the user may enter identifying information for the sample (at 304). The sample may have a scannable code, such as an optically scannable bar code or QR code affixed to it that may be automatically scanned with a camera located on the surface or within the mobile device. Any of a number of identifier labelling techniques, such as radio frequency identifiers (RFIDs) and so on may be used. Alternatively, a unique serial number, code or other identifier associated with the spray tank may be manually entered into the mobile device 18 and transmitted to the server 26. Additionally, the user may use the mobile device to scan in or manually enter one or more substance/analyte identifiers, such as a Universal Product Code (UPC) for the one or more substances, to inform the server of the one or more analytes that the sensor will be providing data on (at 306). The mobile device 18 may also include geolocation information in its communications with the server, either from a GPS sensor included in the mobile device 18 or a GPS software function capable of generating the location of the mobile device in cooperation with a cellular or other communication network in communication with the mobile device.

After authenticating the user and equipment information, and assuming that the server does not identify a mismatch in the probe capability and the type of analyte or substance to be tested, or any other user, device or location authenticity issue, real-time data from the probe and detection circuitry of the detector are transmitted to the mobile device 18. The mobile device 18 transmits the real-time data to the server and the server 26 processes the data in real-time to account for the age of the probe and probe type to determine analyte levels (at 308). The ongoing analyte level measurements may be transmitted back to the mobile device 18 and displayed by the mobile device 18 to the user (at 310).

At any time during detection and monitoring, a signal may be sent to initiate and perform a predictive modeling process for future analyte contamination (312) and, if needed, remedial or preventative measures. The step of performing predictive modeling (312) may include the step of building a predictive model based on the data and various variables described herein. The step of performing predictive modeling (312) may include the application of one or more analytical approaches including, for example, artificial intelligence, APACHE II algorithm, an APACHE III algorithm, Bayesian network, correlation analysis, causal analysis, time series analysis, survival modeling, and machine learning techniques to automatically learn rules and build predictive models based on system measurements. The step of performing predictive modeling (312) may indicate the amount of time required for an analyte to spread amongst or infect an environment or population.

The step of performing predictive modeling (312) may include analyzing data for one or more variables or clusters of variables. The data variables may include data from other analyte detection and monitor systems. The data variables may include personal health data including, but are not limited to, body temperature, age, height, gender, weight, DNA profile, geolocation, vaccine history, general medical history (any or all data from an electronic medical record) or any other variable that pertains to individuals that may exhibit a high degree of analyte infection. The data variables may include environmental data including, but not limited to, prevalence of viruses (e.g., common cold or influenza), allergen levels (e.g., fungi or pollen) or weather data (e.g., humidity, rain fall, wind speed and direction). Other data variables include reproductive number, population density, intra-regional transit frequency, analyte transmission rate, population movement (e.g., people or animals), drinking water analyte, building layout and air circulation, or other variable inherent in movement of analytes in the environment.

The detection of analytes and compilation of the data with modeling allows for confirmation in real time. In addition to coupling multiple detections of a target analyte across a region or at a particular geographic location, detections of other analytes may be used to build and validate the predictive model as well as improve its accuracy. The predictive model may rapidly and continuously update the predictive model as more data is collected and analyzed.

The step of performing predictive modeling (312) may include predicting whether or not a particular individual having characteristics of the randomly generated sample will become infected by the particular analyte. The step of performing predictive modeling (312) may include predicting movement of a particular analyte across a geographic region. The step of performing predictive modeling (312) may include predicting the rate of infection across a population or geographic region.

The step of performing predictive modeling (312) may be undertaken by a predictive modeling system for predictively modeling analyte contamination. The predictive modeling system may utilize one or more processors, servers and databases provided herein. The one or more processors, servers and databases may be located at a location remote to the detection units thereby allowing the predictive model system to analyze data generated by at one or more different locations. Data obtained from the one or more detector units may be processed by one or more processors and stored on one or more servers or databases.

The step of performing predictive modeling (312) may generate an electronic report. The electronic report may be printed for review and distribution The report may be denied or accepted electronically by an end user. The report may include one or more predictions regarding the predicted spread of infection across an environment or population. The report may include one or more recommendations for aiding in the prevention of analyte infection based on the predictive model. The recommendations may be denied or accepted electronically by an end user.

Although the data transfer for the sensed contamination information for the detector 12 may be sent to the server 26 for processing, and the server may then analyze that data to determine analyte level and immediately transmit back the analyte level information and a completion signal to the mobile device 18, in other embodiments, the mobile device may calculate and display the contamination level information and generate the completion signal internally. In this alternative embodiment, the mobile device may still perform the steps of authenticating user ID, detector information, sample identification and analyte identification with the server 26 (steps 302, 304 and 306), but instead of then sending the raw sensed analyte data to the server 26, the mobile device may internally identify and determine the analyte level from the raw sensor data without transmitting it to the server 26. In this alternative embodiment, the algorithms for identifying analyte level, for adjusting calculation based on probe or other detector information and for recognizing the point (e.g. a predetermined analyte level threshold or predetermined analyte level range) when a desired analyte level has been reached may all be completed and generated at the mobile device itself. In order to implement this alternative embodiment, the memory of the mobile device may be pre-loaded with instructions for making the analysis, or the server 26 may transmit to the mobile device the instructions and other information for the mobile device to locally process the data in response to receiving the authentication and device identification information from the mobile device (steps 302-306).

In one alternative embodiment, the mobile device 18, may send a signal preventing operation of any decontamination or cleaning process equipment, if there is a mismatch or other irregularity in the authentication information (user ID, geolocation information, etc.) provided to the server with the information contained in the server. For example, if the server determines from the analyte identifying information and the probe or other sensor identifying information that the probe 20 (or probes) is not suited to test for the analyte, then the server may send a signal notifying the user not to start the process.

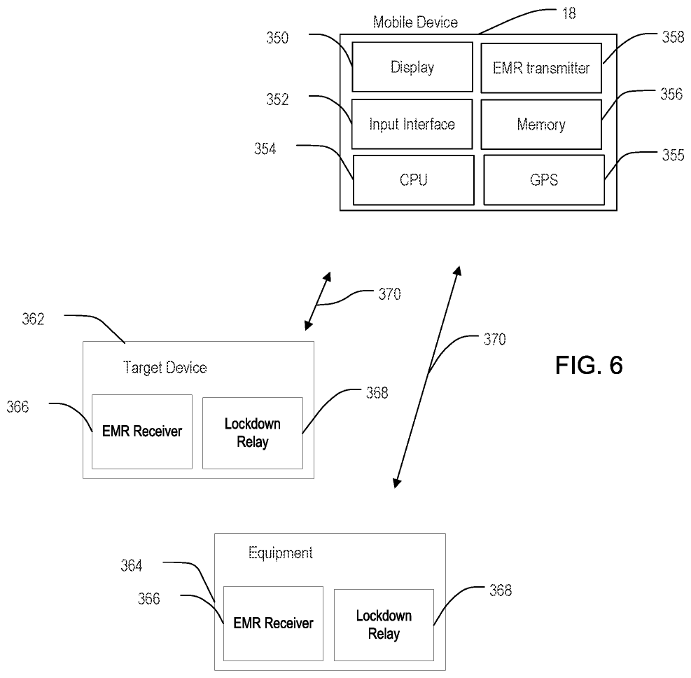

Referring to FIG. 6, in one implementation, an interlock-enabled system and process consists of the detection system 10, for example the mobile device 18 of the detection system 10, having a suitable electromagnetic radiation (EMR) transmitter 358, for example radio frequency, RFID, Wi-Fi, Bluetooth, cellular or optical technologies. The mobile device 18 may be a smartphone, tablet or other portable device having a display 350, user input interface 352, processor 354, GPS location function or sensor 355, memory 356 and one or more EMR transmitters 358. Any piece of equipment controllable by the mobile device 18 may include, either integrated in its circuitry or as a discrete add-on component, an EMR receiver 366 compatible with the EMR transmitter 358, and an EMR-activated relay 368.

The mobile device 18 of the detector system 10 may be programmed in memory 356 to send an EMR signal when sample results are within the specified range as determined locally or by the server. The EMR signal may be a direct wireless communication link 370 between mobile device and equipment 364 or target device 362 as illustrated, or may be via a communication path over one or more networks in communication with the equipment and mobile device 18. Because the EMR receiver 366 is preferably linked to a relay 368 that controls the power to activate the connected equipment upon receipt of the signal, automated control of the particular equipment by the detection system 10 may be achieved. It is contemplated that the equipment that can be included in interlocked mode with the detection system may include shut-off valves, pumps, power control units, motors, and a variety of off/on switches available for industrial processes. Also, it is contemplated that the mobile device 18 would only be able to control the particular piece of equipment located in geographical proximity to the mobile device based on the testing or authentication taking place at the processing stage where the user and mobile device are located. The various different pieces of equipment illustrated in FIG. 6 are representative of the types of equipment the automated shut-down or lockout process may be applied and does not represent that all of these pieces of equipment must either be at the same geographical location or be simultaneously controllable by the shut-off command transmitted by a single mobile device. In another embodiment, the more than one piece of equipment, or more than one part of a single piece of equipment, may be independently and concurrently controlled by remote commands from the mobile device 18.

A management and safety override function or system may be included to release or reset the systems affected by a shutdown. In one implementation, it is contemplated that interlock (lockdown) activation when a analyte level is too high may also trigger the detector system 10 to record the time and GPS location of the initiation and termination of signals for the shutdown. The mobile device 18 may store this locally in memory 356 and/or transmit this information to the server 26. When the interlock is triggered, the mobile device 18 may also concurrently generate and transmit a notification of the interlock activation to a management device or devices. The notification may be an automatically generated call, text, email or other communication and may include the time and location of the shutdown, as well as details on the user and specific equipment affected. If in reply an authorized management signal is subsequently received at the mobile device 18, the shutdown equipment may be released from the interlock shutdown command and resume operation.

An advantage of the mobile device 18 and at least one portable detector 12 is that they can be used on location to send real-time data from the probe or probes to a server, such as a remote server, for interpretation in real-time. Alternatively, the real-time data from the probe(s) may be interpreted and processed locally at the mobile device to provide analyte level. A plurality of portable detectors 12 may be utilized to work together across a geographic region, city, county, state, country, or around the globe.

As described previously, the probe 20 and detector circuitry 22 of a portable detector 12 that may be used in the detection system 10 described herein may be configured for measuring the presence of one or of multiple different analytes. Due to the ability of the detection system to detect and transmit information in real-time, difficult to distinguish substances such as viruses and bacterium may be more successfully differentiated. Signals from different probes may be combined in the present system to allow the computation layer of the server to interpret the signals in real-time using a comparison algorithm based on predetermined operating characteristics of the particular probe or probe technology.

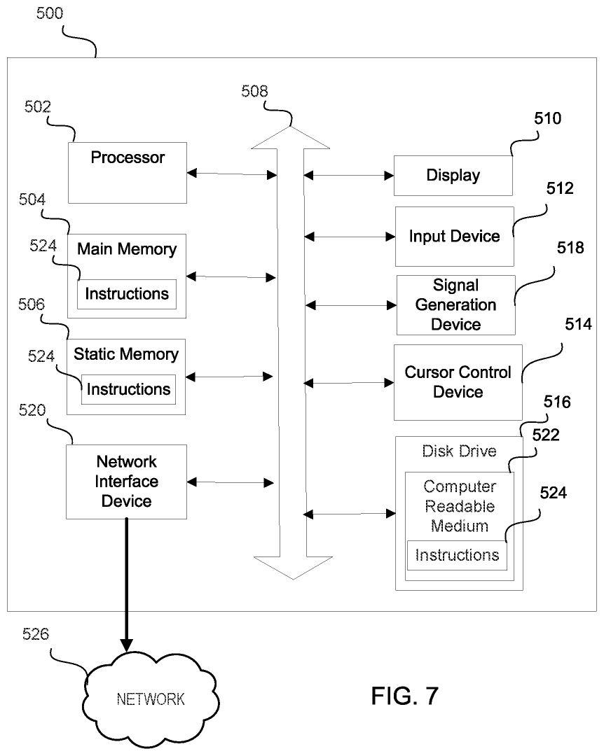

Referring to FIG. 7, an illustrative embodiment of a general computer system that may be used in, or for, one or more of the components described above, or in any other system configured to carry out the methods discussed above, is shown and is designated 500. The computer system 500 can include a set of instructions that can be executed to cause the computer system 500 to perform any one or more of the methods or computer-based functions disclosed herein. The computer system 500 may be mobile or non-mobile, operate as a stand-alone device, or may be connected using a network, to other computer systems or peripheral devices.

In a networked deployment, the computer system may operate in the capacity of a server or as a client user computer in a server-client user network environment, or as a peer computer system in a peer-to-peer (or distributed) network environment. The computer system 500 can also be implemented as, or incorporated into, various devices, such as a personal computer (“PC”), a tablet PC, a set-top box (“STB”), a personal digital assistant (“PDA”), a mobile device such as a smart phone or tablet, a palmtop computer, a laptop computer, a desktop computer, a network router, switch or bridge, or any other machine capable of executing a set of instructions (sequential or otherwise) that specify actions to be taken by that machine. In a particular embodiment, the computer system 500 can be implemented using electronic devices that provide voice, video or data communication. Further, while a single computer system 500 is illustrated, the term “system” shall also be taken to include any collection of systems or sub-systems that individually or jointly execute a set, or multiple sets, of instructions to perform one or more computer functions.

As illustrated in FIG. 7, the computer system 500 may include a processor 502, such as a central processing unit (“CPU”), a graphics processing unit (“GPU”), or both. Moreover, the computer system 500 can include a main memory 504 and a static memory 506 that can communicate with each other via a bus 508. As shown, the computer system 500 may further include a video display unit 510, such as a liquid crystal display (“LCD”), an organic light emitting diode (“OLED”), a flat panel display, a solidstate display, signal lights, or a cathode ray tube (“CRT”). Additionally, the computer system 500 may include one or more input devices 512, such as a keyboard, scanner, digital camera or audio input device, and a cursor control device 514, such as a mouse. The computer system 500 can also include a memory unit 516, which may be a solid state or a disk drive memory, a signal generation device 518, such as a speaker or remote control, and a network interface device 520.

In a particular embodiment, as depicted in FIG. 7, the memory unit 516 may include a computer-readable medium 522 in which one or more sets of instructions 524, such as software, can be embedded. Further, the instructions 524 may embody one or more of the methods or logic as described herein. In a particular embodiment, the instructions 524 may reside completely, or at least partially, within the main memory 504, the static memory 506, and/or within the processor 502 during execution by the computer system 500. The main memory 504 and the processor 502 also may include computer-readable media.

In an alternative embodiment, dedicated hardware implementations, including application specific integrated circuits, programmable logic arrays and other hardware devices, can be constructed to implement one or more of the methods described herein. Applications that may include the apparatus and systems of various embodiments can broadly include a variety of electronic and computer systems. One or more embodiments described herein may implement functions using two or more specific interconnected hardware modules or devices with related control and data signals that can be communicated between and through the modules, or as portions of an application-specific integrated circuit. Accordingly, the present system encompasses software, firmware, and hardware implementations.

In accordance with various embodiments of the present disclosure, the methods described herein may be implemented by software programs executable by a computer system. Further, in an exemplary, non-limited embodiment, implementations can include distributed processing, component/object distributed processing, and parallel processing. Alternatively, virtual computer system processing can be constructed to implement one or more of the methods or functionalities as described herein.

The present disclosure contemplates a computer-readable medium that includes instructions 524 or receives and executes instructions 524 responsive to a propagated signal; so that a device connected to a network 526 can communicate voice, video or data over the network 526. Further, the instructions 524 may be transmitted or received over the network 526 via the network interface device 520.

While the computer-readable medium is shown to be a single medium, the term “computer-readable medium” includes a single medium or multiple media, such as a centralized or distributed database, and/or associated caches and servers that store one or more sets of instructions. The term “computer-readable medium” shall also include any tangible medium that is capable of storing, encoding or carrying a set of instructions for execution by a processor or that cause a computer system to perform any one or more of the methods or operations disclosed herein.

In a particular non-limiting, exemplary embodiment, the computer-readable medium can include a solid-state memory such as a memory card or other package that houses one or more non-volatile read-only memories, such as flash memory. Further, the computer-readable medium can be a random access memory or other volatile re-writable memory. Additionally, the computer-readable medium can include a magneto-optical or optical medium, such as a disk or tapes or other storage device to capture information communicated over a transmission medium. A digital file attachment to an e-mail or other self-contained information archive or set of archives may be considered a distribution medium that is equivalent to a tangible storage medium. Accordingly, the disclosure is considered to include any one or more of a computer-readable medium or a distribution medium and other equivalents and successor media, in which data or instructions may be stored.

Although the present specification describes components and functions that may be implemented in particular embodiments with reference to particular standards and protocols commonly used by financial institutions, the invention is not limited to such standards and protocols. For example, standards for Internet and other packet switched network transmission (e.g., TCP/IP, UDP/IP, HTML, HTTP) represent examples of the state of the art. Such standards are periodically superseded by faster or more efficient equivalents having essentially the same functions. Accordingly, replacement standards and protocols having the same or similar functions as those disclosed herein are considered equivalents thereof.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

### Prophetic Example 1

### Viral Detection, Quantification and Modeling System

The system and methods as provided herein may be utilized to collect, analyze and detect one or more viruses in the air at a chosen location (either indoors or outdoors). Typical locations to be monitored could include but are not limited to transportation hubs (airports, bus stations, border crossings), schools, places of business, medical facilities, labs, government buildings, and sporting events. Such systems and methods may utilize one or more systems and methods as provided herein. The one or more detectors may provide real-time data regarding the presences of one or more viruses in the air at the location. Such detectors may continue to operate in a monitoring function. If a certain level of analyte is detected, a signal may be sent to an appropriate authority (e.g., Department of Homeland Security; CDC). The system may perform predictive modeling to predicting movement of a particular analyte across a geographic region based on data obtained from an array of detectors at one or more locations as noted herein. The system may predict the rate of infection across a population or geographic region based on data obtained from the array of detectors. Such a system would be helpful in signaling a potential pandemic threat and implementing remedial and preventative measures. Particularly, the system and method will aid in preventing further spread of the virus to the animal/human and even plant population.

By way of particular example, if a detector signaled a positive result for SARS-CoV-2 at a transportation hub in Fort Wayne, Ind., the predictive modeling system could incorporate evidence of other positive detections in northern Indiana and the surrounding region via a networked array of detectors described herein and prepare a predictive model of how the SARS-CoV-2 infection might spread across the state of Indiana and surrounding states.

### Prophetic Example 2

### Analyte Detection, Quantification and Modeling in Dental Offices

The systems provided herein may be utilized to aid in high throughput quantification, monitoring and predictive modeling of analytes such as viruses (e.g., SARS-CoV-2) via implementation of systems in dental offices. Dental offices deal with several oral diseases but are also subject to common disease and viral threats such as HIV, Hepatitis, Flu, and Corona Viruses such as SARS-CoV-2. The United States dental industry has over 150,000 dental hygienists which see roughly 8 patients a day or roughly 1,200,000 per day nationwide (0.38% of United States population daily or approximately 7.5% of population monthly). More than half of the United States population visits a dental hygienist at least once per year. Dental hygienists are trained to deal with both saliva and blood and the real potential that the patient could be contagious with various analytes such as SARS-CoV-2. Using the systems provided herein to detect for these potential viral analytes prior to a dental exam can serve at least three purposes: (i) prevent transmission to the dental worker of other professional; (ii) diagnose a patient while providing early intervention; and (iii) monitor analytes to help prevent outbreak, epidemic, or pandemic.

According to one embodiment, the systems provided herein may be utilized to screen or otherwise detect an analyte for each patient prior to or upon entering a dental office (HIPAA compliance required). The system may be located in a lobby or separate area such that results regarding analyte infection may be provided prior to entry into the office and subsequent dental treatment. Screening may occur with a saliva or blood sample from a patient. Such a screening process may be financially subsidized by a patient's dental insurance as well as supported by both the ADA and the AMA. According to one embodiment, the systems provided herein may be utilized to provide the dental office with an additional source of revenue via patient screening.

According to one embodiment, the systems provided herein may be utilized to monitor the rinse water from a dental “rinse” sink. The results of such monitoring may be sent to a third monitoring service. According to such an embodiment, the system provides a reliable sampling of the general United States population. Since the sampling device is connected to a rinse sink, the water collected is less variable and more reliable for the sampler allowing for simpler design. By enumerating the patients, geolocating the sink, and sending the data to a central location (e.g., cloud-based server), the system may function as a digitized monitoring system for mapping results across a geographic location.

According to another embodiment, the systems provided herein may be utilized to monitor the rinse water from a dental suction line used during a dental cleaning or procedure. The results of such monitoring may be sent to a third monitoring service. According to such an embodiment, the system provides a reliable sampling of the general population in a city, county state or country. Since the sampling device is connected to a suction line, the water collected is less variable and more reliable for the sampler allowing for simpler design. By enumerating the patients, geolocating the suction line, and sending the data to a central location (e.g., cloud-based server), the system may function as a digitized monitoring system for mapping results across the United States. After sampling rinse water from a particular patient, the system may immediately dispense test results to public health organizations or other authority to facilitate rapid predictive modeling and effective response recommendations to minimize the analyte impact. The test results may include geolocation data as well as patient or target sample identity to facilitate contact tracing. According to a particular embodiment, the cartridge within the system may then be removed and replaced with a new cartridge or cleaned prior to next use.

The systems provided herein can also become part of the normal maintenance of those managing the dental office making the detection and monitoring method seamless. The statistical relevance of this type of monitoring allows for non-HIPAA collection of data while also allow forming monitoring and predictive modeling of the health of a particular region and, in turn, the overall country. In the event medical privacy laws (e.g., HIPAA laws) require authorization for this type monitoring, a bypass switch can be installed and the system will reduce the sample size for analysis by that number.

### Prophetic Example 3

### Analyte Detection, Quantification and Modeling in Industrial Food Processing Applications

The systems provided herein may be utilized to aid in high throughput detection, quantification, monitoring and predictive modeling of pathological analytes in industrial applications such as in food processing (and packaging) plants. Particularly, a system as provided herein may be used in conjunction with a piece of processing equipment or vessel to detect, quantify and monitor pathological analytes. A system as provided herein may be used to detect, quantify and monitor, for example, aflatoxin levels during peanut butter production or melamine during milk production. A system as provided herein may be used to detect, quantify monitor, and predictively model any bacteria or fungus present during food processing.

The systems provided herein may be also utilized with an automatic interlock system that shuts down food production upon detection of a pathological analyte at a certain level. Using the systems provided herein to detect for these pathological analytes can prevent food spoilage and contamination thereby also preventing food-borne illness by the consumer. The systems provided herein may also initiate a decontamination procedure to remove the pathological analyte from the equipment or vessel prior to resuming food production.

The systems provided herein may be also utilized to predictively model the spread or infection of a population by one or more pathological analytes present in food.

The systems provided herein may be also utilized to send collected data regarding pathological analyte presence and levels to a monitoring service. According to such an embodiment, the system provides a reliable record of the data that could be accessed by interested third parties such as insurance companies or downstream consumers of the food produced.

### Prophetic Example 4

### Analyte Detection, Quantification and Modeling Via Medical Diagnostics

The systems provided herein may be utilized to aid in high throughput detection, quantification, monitoring and predictive modeling of pathological analytes based on analyte testing data obtained from an array of detectors or from direct input from third party medical diagnostics. Particularly, an array of detectors may be utilized at various healthcare settings or mobile testing units that aid in high throughput detection and quantification of any one or more analytes such as avian influenza or SARS-CoV-2.

The systems provided herein may be also utilized to predictively model the spread or infection of an analyte such as avian influenza or SARS-CoV-2.

The systems provided herein may be also utilized to send collected data regarding pathological analyte presence and levels to a monitoring service. According to such an embodiment, the system provides a reliable record of the data that could be accessed by interested third parties such as the CDC or a government agency.

### Prophetic Example 5

### Analyte Detection, Quantification and Modeling in Water Treatment Industry

The systems provided herein may be utilized to aid in high throughput detection, quantification, monitoring and predictive modeling of pathological analytes in industrial water treatment applications. Particularly, a system as provided herein may be used in conjunction with municipal water supplies (sewage treatment), wells, bodies of water such as lakes and ponds, groundwater or other water storage and treatment facilities to detect, quantify, monitor, and predictively model pathological analyte spread or future contamination. A system as provided herein may be used to detect, quantify and monitor, for example, nitrogen, chemicals (e.g., agricultural chemicals such herbicides, fertilizers or pesticides), metals, or any pathogen as provided herein.

The systems provided herein may be also utilized with an automatic interlock or safety system that shuts down water treatment upon detection of a pathological analyte at a certain level in water that is believed to be acceptable for human consumption or use. Using the systems provided herein to detect for these pathological analytes can prevent water-borne illness by the consumer. The systems provided herein may also initiate a decontamination procedure to remove the pathological analyte from the industrial water treatment equipment water itself prior to resuming water treatment.

The systems provided herein may be also utilized to predictively model the spread or infection of a population by one or more pathological analytes present in water.

The systems provided herein may be also utilized to send collected data regarding pathological analyte presence and levels to a monitoring service. According to such an embodiment, the system provides a reliable record of the data that could be accessed by interested third parties such as state, federal or municipality-level authority that oversee water quality.

### Prophetic Example 6

### Analyte Detection, Quantification and Modeling on Livestock Farms

The systems provided herein may be utilized to aid in high throughput detection, quantification, monitoring and predictive modeling of pathological analytes on livestock farms. Particularly, a system as provided herein may be used in conjunction with a piece of processing equipment or vessel to detect, quantify and monitor pathological analytes present amongst livestock such as hogs or chickens. A system as provided herein may be used to detect, quantify, monitor, and predictively model for example, common pathological analytes in livestock such as Asian swine flu or avian influenza.

Using the systems provided herein to detect for these pathological analytes can food-borne illness by the consumer. The systems provided herein may also initiate a modeling step to predictively model the spread or infection of a population by one or more pathological analytes present in or around livestock.

The systems provided herein may be also utilized to send collected data regarding pathological analyte presence and levels to a monitoring service. According to such an embodiment, the system provides a reliable record of data that could be accessed by interested third parties.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

We claim:

1. An analyte detection, monitoring and modeling system comprising:
at least one portable detector having a probe and detector circuitry for detecting an analyte in a sample and producing analyte detection data; and
at least one mobile device configured to wirelessly receive the analyte detection data from the portable detector and transmit the analyte detection data from the portable detector to a processor in real-time, wherein the processor is configured to:
quantify a level of analyte;
monitor the level of analyte; and
display, in real-time, on the mobile device all data related to type and level of analyte present; and execute a predictive modeling system.

2. The analyte detection, monitoring and modeling system of claim 1, wherein the detector, mobile device and predictive modeling system are integrated into a single, mobile unit sized to be hand-held.

3. The analyte detection, monitoring and modeling system of claim 2, wherein the mobile device is selected from the group consisting of a smartphone, tablet, and portable computer.

4. The analyte detection, monitoring and modeling system of claim 1, wherein the at least one portable detector comprises a plurality of probes, each of the probes configured to detect a different analyte.

5. The analyte detection, monitoring and modeling system of claim 1, wherein the at least one portable detector includes a collection apparatus configured to receive a target sample.

6. The analyte detection, monitoring and modeling system of claim 3, wherein the mobile device is configured to:
wirelessly receive analyte detection data from the at least one portable detector for each of the plurality of probes and to transmit the analyte detection data from the at least one portable detector to a processor in real-time; and
receive and display on the mobile device real-time analyte level data for each of a plurality of different analytes determined by the remote processor from the analyte detection data for the plurality of probes.

7. The analyte detection, monitoring and modeling system of claim 1, wherein the mobile device is configured to transmit a signal to initiate predictive modeling via the predictive modeling system.

8. The analyte detection, monitoring and modeling system of claim 7, wherein the predictive modeling system receives real-time analyte level data from the mobile device.

9. The analyte detection, monitoring and modeling system of claim 8, wherein the mobile device is configured to receive analyte detection data from two or more portable detectors.

10. The analyte detection, monitoring and modeling system of claim 8, wherein the predictive modeling system analyzes data for one or more variables selected from the group consisting of personal health data, environmental data, weather data, analyte transmission rate, movement of vectors/carriers, building layout and air circulation.

11. A method of determining the level of analyte in a target sample and modeling future contamination, the method comprising the steps of:
introducing a probe of at least one portable detector system to a sample, wherein the probe is configured to detect at least one analyte in the sample;
wirelessly transmitting analyte detection signals from detection circuitry in communication with the probe to a mobile device of the at least one portable detector system;
transmitting, in real-time, the analyte detection signals from the mobile device to a processing system;
receiving, in response to the transmitted analyte detection signals, real-time analyte level data processed by the processing system from the analyte detection signals;
displaying the real-time analyte level data to a user on a display of the mobile device; and
predictively modeling future analyte spread, infection or contamination.

12. The method of claim 11, wherein the at least one portable detector system comprises a plurality of probes, each of the probes configured to detect a different analyte; and
wherein the method further comprises concurrently transmitting analyte detection signals from detection circuitry in communication with each of the plurality of probes to the mobile device of the at least one portable detector system.

13. The method of claim 11, further comprising the step of transmitting any modeling data to a third party capable of implementing remedial or preventative measures against the analyte contamination.

14. The method of claim 11, further comprising the step of producing a report including a prediction regarding future analyte spread, infection or contamination.

15. The method of claim 14, wherein the report includes one or more recommendations for preventing future analyte spread, infection or contamination.

16. The method of claim 11, further comprising the step of denying or accepting the one or more recommendations by a user.

17. The method of claim 11, wherein the sample is taken from a surface, air, human or animal.

18. The method of claim 11, wherein the sample is taken from a public or private space selected from the group consisting of a food processing facility, healthcare facility, airport, train station, border crossing, and office space.

19. A kit comprising an analyte detection, monitoring and modeling system of claim 1 and at least one set of instructions.
